(19) United States
(12) Patent Application Publication
(10) Pub. No.: US 2022/0165435 A1
(43) Pub. Date: May 26, 2022

(54) DRUG REPOSITIONING CANDIDATE RECOMMENDATION SYSTEM, AND COMPUTER PROGRAM STORED IN MEDIUM IN ORDER TO EXECUTE EACH FUNCTION OF SYSTEM

(71) Applicant: KOREA INSTITUTE OF SCIENCE & TECHNOLOGY INFORMATION, Daejeon (KR)

(72) Inventor: Hyo Jung PAIK, Daejeon (KR)

(21) Appl. No.: 17/440,625

(22) PCT Filed: Mar. 31, 2020
(86) PCT No.: PCT/KR2020/004431

§ 371 Date: Sep. 17, 2021

(30) Foreign Application Priority Data
Apr. 1, 2019 (KR) 10-2019-0037940

(51) Int. Cl. G16H 70/40; G16B 20/00

(52) U.S. Cl. CPC G16H 70/40; G16B 20/00

(57) ABSTRACT

The present disclosure relates to a technology capable of utilizing literature information and genomic signatures, which is a large amount of big data, so as to predict a new indication of a drug of which the safety has been verified, and recommend a drug repositioning candidate according to the prediction result.

### TECHNICAL FIELD

The present disclosure relates to a technology for recommending a new drug repositioning candidate.

### BACKGROUND

Recently, multinational pharmaceutical companies are facing a significant crisis of worsening profitability due to an increase in new drug development costs.

In order to overcome this crisis, there is a need for a low-cost/high-efficiency new drug development method, and drug repositioning is drawing attention as a new method for satisfying this need.

Drug repositioning is a method for re-evaluating a drug that is being used in clinical trials or is in commercial use so as to find a new medical effect, and there is a higher chance of success since the safety of the drug to be developed has already been verified to a certain extent.

Most successful drug repositioning cases in the clinical trial field are from accidental discoveries of new indications in the process of pre-clinical trials or treatment. However, in recent times, various drug screening and drug evaluation technologies have been developed, and thus, more systematic drug repositioning can be made according to identification of a disease association target gene.

Specifically, as production of a large amount of gene expression data (hereinafter, referred to as “genomic signatures”) is normalized, and various types of disease (or illness)-drug genetic association (response) data are discovered, attempts to study the possibility of inferring a new drug repositioning candidate through mining of the disease-drug genetic association (response) data have been made recently.

Drug repositioning candidate investigation research based on various research techniques such as DNA microarray and biological database mining has been recognized as a main research issue in the bioinformatics field, but there are practical limitations in the research due to difficulties associated with a lack of human resources for research in the field of biological data integrated analysis and an absence of sufficient drug- and disease-associated clinical trial data.

### SUMMARY

The present disclosure is to directed to predicting a new indication of a drug, the safety of which has been verified, and recommending a drug repositioning candidate according to the result of the prediction, without utilizing data which is inevitably restricted, such as physiological information collected from human-derived materials of actual patients, or symptom information or personal medical information protected by laws relating to personal information.

In accordance with a first aspect of the present disclosure, a drug repositioning candidate recommendation system includes: an extraction unit configured to extract character information of a drug and a disease on the basis of open literature information, and extract genetic association information of a drug and a disease on the basis of genomic signatures; a first matrix configuration unit configured to configure a drug-drug or a disease-disease similarity matrix on the basis of the information extracted from the literature information; a second matrix configuration unit configured to configure a drug-drug or a disease-disease similarity matrix on the basis of the information extracted from the genomic signatures; a calculation unit configured to calculate a literature information-based drug-disease edge score (P_t) according to the similarity matrix configured by the first matrix configuration unit, and calculate a genomic signature-based drug-disease edge score (P_g) according to the similarity matrix configured by the second matrix configuration unit; and a recommendation unit configured to recommend a drug repositioning candidate according to a value determined by using at least one of the calculated score (P_t) or the calculated score (P_g).

In accordance with a second aspect of the present disclosure, a computer program stored in a medium so as to execute, in combination with hardware, the following operations including: an information extraction operation of extracting character information of a drug and a disease on the basis of open literature information, and extracting genetic association information of a drug and a disease on the basis of genomic signatures; a first matrix configuration operation of configuring a drug-drug or a disease-disease similarity matrix on the basis of the information extracted from the literature information; a second matrix configuration operation of configuring a drug-drug or a disease-disease similarity matrix on the basis of the information extracted from the genomic signatures; a calculation operation of calculating a literature information-based drug-disease edge score (P_t) according to the similarity matrix configured in the first matrix configuration operation, and calculating a genomic signature-based drug-disease edge score (P_g) according to the similarity matrix configured in the second matrix configuration operation; and a recommendation operation of recommending a drug repositioning candidate according to a value determined by using at least one of the calculated score (P_t) or the calculated score (P_g).

Specifically, the recommendation operation may include a final calculation operation of calculating a final prediction score f(e_ij) of a drug-disease edge by using the calculated score (P_t) and the calculated score (P_g); and a recommendation operation of recommending a drug repositioning candidate according to a value determined with reference to the final prediction score f(e_ij).

Specifically, the literature information may include at least one of: academic articles and medical or pharmaceutical books including description of symptoms of a disease, drug administration information, and description of a drug responsive character, a drug indication, or an adverse drug effect; an open database in which computational technology-based drug and disease association character information is collected; or disease and drug association description information.

Specifically, the first matrix configuration operation may include: configuring an association word vector which indicates an appearance frequency of an association character word as an information value for each drug on the basis of the character information of a drug, the information being extracted from the literature information; and configuring a drug-drug similarity matrix by calculating a cosine similarity between association word vectors of respective drugs on the basis of the association word vector of each drug.

Specifically, the first matrix configuration operation may include: configuring an association word vector which indicates an appearance frequency of an association character word as an information value for each disease on the basis of the character information of a disease, the information being extracted from the literature information; and configuring a disease-disease similarity matrix by calculating a cosine similarity between association word vectors of respective diseases on the basis of the association word vector of each disease.

Specifically, an information value in the association word vector of the drug or an information value in the association word vector of the disease may be defined as t_ij indicating an appearance frequency of an i-th association character word of a j-th drug or a j-th disease, and the information value (t_ij) may be a value obtained by normalizing a frequency count (T_ij) of appearances of the i-th association character word in one piece of literature to a frequency count (n_i) of appearances of the i-th association character word in all of the literature information.

Specifically, a network configuration operation of configuring a drug-disease bipartite network on the basis of drug indication information may be further included, and the calculation operation may include: calculating a literature information-based drug-disease edge score (P_t) by using the similarity matrix configured in the first matrix configuration operation and the configured drug-disease bipartite network; and with respect to a pair of a particular drug (s_i, an i-th drug) and a particular disease (t_j, a j-th disease), calculating a drug-disease edge score (P_t) by using a similarity value between the particular drug (s_i) identified from the drug-drug similarity matrix configured in the first matrix configuration operation and a reference drug (s_p) selected for calculation, a similarity value between the particular disease (t_j) identified from the disease-disease similarity matrix configured in the first matrix configuration operation and a reference disease (t_q) selected for calculation, an edge between the reference drug (s_p) and the reference disease (t_q), and a degree value of the reference drug (s_p) identified from the drug-disease bipartite network.

Specifically, the reference drug (s_p) may be selected with reference to a pre-verified similarity to the particular drug (s_i), and the reference disease (t_q) having a true value of an edge label with the reference drug (s_p) from pre-verified drug-disease association relationships may be selected, or the reference disease (t_q) may be selected with reference to a pre-verified similarity to the particular disease (t_j), and the reference drug (s_p) having a true value of an edge label with the reference disease (t_q) from pre-verified drug-disease association relationships may be selected.

Specifically, the final calculation operation may include: identifying heritability with respect to a pair of a particular drug (s_i) and a particular disease (t_j) used to calculate the score (P_t) and the score (P_g); and calculating the final prediction score f(e_ij) of the drug-disease edge by using different schemes according to the heritability.

Specifically, the final calculation operation may include: when the heritability has a value equal to or larger than a predefined reference value, calculating the final prediction score f(e_ij) of the drug-disease edge by giving a larger weight to the genomic signature-based drug-disease edge score (P_g) than to the score (P_t); and when the heritability has a value smaller than the reference value, calculating the final prediction score f(e_ij) of the drug-disease edge by giving a larger weight to the literature information-based drug-disease edge score (P_t) than to the score (P_g).

Specifically, the recommendation operation may include: determining a true or false value according to a reference value (cut-off); and when the value is true, identifying a pair of a particular drug (s_i) and a particular disease (t_j) used to calculate the final prediction score f(e_ij) so as to recommend the particular drug (s_i) as a new drug for the particular disease (t_j).

According to embodiments of the present disclosure, a new type of drug repositioning candidate recommendation technique (technology) capable of predicting a new indication of a drug, the safety of which has been verified, and recommending a drug repositioning candidate according to the result of the prediction, without utilizing data which is inevitably restricted due to the lack of human resources and the characteristics of physiological information collected from human-derived materials of actual patients or symptom information or personal medical information protected by laws relating to personal information, can be implemented.

According to the present disclosure, predicting a new indication of a drug, the safety of which has been verified, and recommending the drug are possible through various drug- and disease-associated academic articles/literature information and genomic signatures that have been accumulated to date, whereby significant reduction in drug development duration and cost can be expected.

### BRIEF DESCRIPTION OF DRAWINGS

FIG. 1 illustrates a configuration of a drug repositioning candidate recommendation system according to an embodiment of the present disclosure.

FIG. 2 illustrates a process of configuring a drug-disease bipartite network according to the present disclosure.

FIG. 3 is a flow chart illustrating a drug repositioning candidate recommendation technique executed by a computer program according to an embodiment of the present disclosure.

### BEST MODE FOR CARRYING OUT THE INVENTION

Hereinafter, embodiments of the present disclosure are described with reference to accompanying drawings.

The present disclosure relates to the technical field of drug repositioning.

Drug repositioning is a method for re-evaluating a drug that is being used in clinical trials or is in commercial use so as to find a new medical effect, and there is a higher chance of success since the safety of the drug to be developed has already been verified to a certain extent.

Most successful drug repositioning cases in the clinical trial field are from accidental discoveries of new indications in the process of pre-clinical trials or treatment. However, in recent times, various drug screening and drug evaluation technologies have been developed, and thus, more systematic drug repositioning can be made according to identification of a disease association target gene.

Specifically, as production of a large amount of gene expression data (hereinafter, referred to as “genomic signatures”) is normalized, and various types of disease (or illness)-drug genetic association (response) data are discovered, attempts to study the possibility of inferring a new drug repositioning candidate through mining of the disease-drug genetic association (response) data have been made recently.

Drug repositioning candidate investigation research based on various research techniques such as DNA microarray and biological database mining has been recognized as a main research issue in the bioinformatics field, but there are practical limitations in the research due to difficulties associated with a lack of human resources for the research in the field of biological data integrated analysis and an absence of sufficient drug- and disease-associated clinical trial data.

Accordingly, the present disclosure proposes a new type of drug repositioning candidate recommendation technique (technology) capable of predicting a new indication of a drug, the safety of which has been verified, and recommending of a drug repositioning candidate according to the result of the prediction, without utilizing data which is inevitably restricted, such as physiological information collected from human-derived materials of actual patients or symptom information or personal medical information protected by laws relating to personal information.

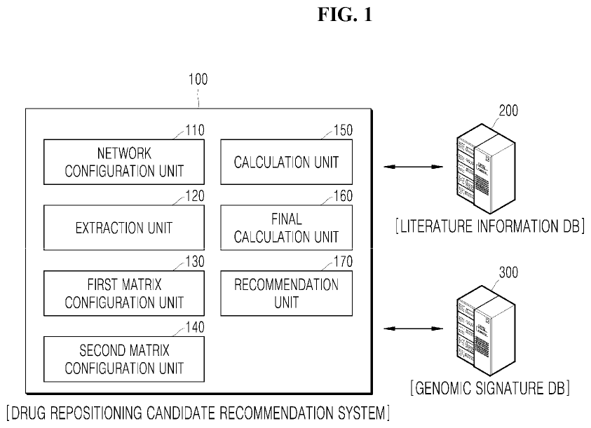

FIG. 1 illustrates a configuration of a drug repositioning candidate recommendation system which implements a drug repositioning candidate recommendation technique (technology) proposed by the present disclosure.

Referring to FIG. 1, a drug repositioning candidate recommendation system 100 of the present disclosure includes an extraction unit 120, a first matrix configuration unit 130, a second matrix configuration unit 140, a calculation unit 150, and a recommendation unit 170.

Furthermore, the drug repositioning candidate recommendation system 100 of the present disclosure may further include a network configuration unit 110 and a final calculation unit 170.

All or a part of the elements of the drug repositioning candidate recommendation system 100 may be implemented as a hardware module, a software module, or a combination of a hardware module and a software module.

Here, the software module may be understood as, for example, an instruction executed by a processor configured to control operations in the drug repositioning candidate recommendation system 100, and such an instruction may be mounted in a memory in the drug repositioning candidate recommendation system 100.

The drug repositioning candidate recommendation system 100 according to an embodiment of the present disclosure may implement, according to the above-described configuration, a technology proposed by the present disclosure, that is, a new type of drug repositioning candidate recommendation technique (technology) capable of predicting the new indication of a drug, the safety of which has been verified, and recommending of a drug repositioning candidate according to the result of the prediction, without utilizing data which is inevitably restricted, such as physiological information collected from human-derived materials of actual patients or symptom information or personal medical information protected by laws relating to personal information.

Hereinafter, each technical element of the drug repositioning candidate recommendation system 100 for implementing the new type of drug repositioning candidate recommendation technique (technology) proposed by the present disclosure will be described in detail.

The network configuration unit 110 may perform a function of configuring a drug-disease bipartite network on the basis of drug indication information.

Specifically, the network configuration unit 110 may configure a drug-disease bipartite network by modeling already known/verified drug indication information, that is, drug-disease association relationships, as a bipartite network.

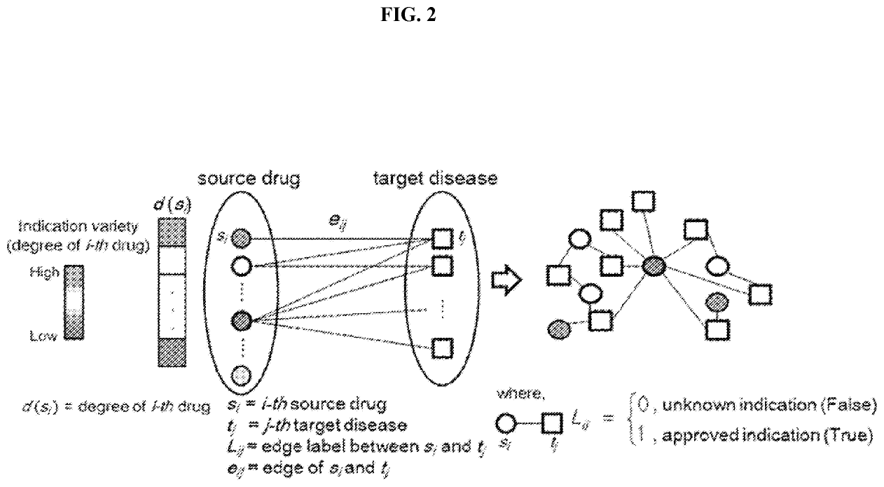

FIG. 2 illustrates a conceptual example of a process of configuring a drug-disease bipartite network in the present disclosure.

The network configuration unit 110 may configure a drug-disease bipartite network defined by a set E={e_11, . . . , e_ij, . . . , e_mn} of N_s, N_t, and e_ij by modeling drug-disease association relationships as a bipartite network.

As shown in FIG. 2, the drug-disease bipartite network configured by the network configuration unit 110 may be represented according to the following concepts.

N_s={s1, s2, . . . , sm}

Here, when the i-th drug among known drugs is indicated as s_i, N_s means a set of all of the known drugs.

N_t={t1, t2, . . . , tn}

Here, when the j-th disease among the known diseases is indicated as t_j, N_t is a set of all of the known diseases.

e_ij indicates an edge for connecting the drug s_i and the disease t_j.

The e_ij is defined by a true or a false value according to a label property, the e_ij may have a value defined by L(e_ij) (0=False or 1=True), and a weight value W(e_ij) satisfying 0<=W(e_ij)<=1 may be added according to the reliability of the association relationships between s_i and t_j. The W(e_ij) information may be configured through the literature information, and the application of the W(e_ij) weight is not essential.

As described above, the network configuration unit 110 may configure a drug-disease bipartite network defined by a set E={e_11, . . . , e_ij, . . . , e_mn} of N_s, N_t, and e_ij on the basis of the known/verified drug-disease association relationships (bipartite network modeling).

The extraction unit 120 performs a function of extracting character information of a drug and a disease on the basis of open literature information, and extracting genetic association information of a drug and a disease on the basis of genomic signatures.

Specifically, the extraction unit 120 extracts character information of a drug and a disease from literature information, which is a large amount of big data, on the basis of association with a literature information DB 200.

Here, the literature information may include at least one of: academic articles and medical or pharmaceutical books including description of symptoms of a disease, drug administration information, and description of a drug responsive character, a drug indication, or an adverse drug effect; an open database in which computational technology-based drug and disease association character information is collected; or disease and drug association description information.

The extraction unit 120 may extract character information (an indication, an adverse effect, or a clinical phenotype) of a drug and a disease from a large amount of literature and bibliographic data such as academic articles, medical or pharmaceutical books, and disease- and drug-associated descriptive information.

The extraction unit 120 may extract genetic association information of a drug and a disease from genomic signatures, which are a large amount of big data, on the basis of association with a genomic signature DB 300.

The extraction unit 120 may collect and extract genetic association information (omics genomic information) from various large-scale genomic signatures (e.g., DrugBank, STITCH, OMIM, etc.) related to the drug and the disease.

The first matrix configuration unit 130 performs a function of configuring a drug-drug or a disease-disease similarity matrix on the basis of information extracted from literature information.

The first matrix configuration unit 130 configures a drug-drug or a disease-disease similarity matrix on the basis of the character information of the drug and the disease, the information being extracted from the literature information by the extraction unit 120.

Specifically, the first matrix configuration unit 130 configures an association word vector which indicates an appearance frequency of an association character word as an information value for each drug on the basis of the character information of the drug, the information being extracted from the literature information.

In addition, the first matrix configuration unit 130 may configure a drug-drug similarity matrix by calculating a cosine similarity between association word vectors of respective drugs on the basis of the association word vector of each drug.

Specifically, the first matrix configuration unit 130 may configure an association word vector for each drug on the basis of the character information of the drug, the information being extracted from the literature information, and, for example, the association word vector (T_dj) of the j-th drug (dj) may be represented as below.

T_dj={t_1j, t_2j, . . . , t_ij, . . . , t_nj}

Here, a value of t_ij indicates an information value in the association word vector of the drug (dj), and is defined to indicate an appearance frequency of the i-th association character word with respect to the drug (dj).

In this case, the information value (t_ij) in the association word vector is defined as a value obtained by normalizing a frequency count (T_ij) of the use (or appearances) of the i-th association character word of the drug (dj) in one piece of literature to a frequency count (n_i) of the use (or appearances) of the i-th association character word in all of the literature information, and may be represented according to Equation 1 below.

$${D}_{k} ( {t}_{ij} ) = \frac{{D}_{k} ( {T}_{ij} )}{{D}_{k} ( {n}_{i} )} \qquad [ Equation 1 ]$$

The information value (e.g., t_ij) in the association word vector of each drug may be defined as an appearance frequency (value) obtained by normalizing the appearance frequency of the association character word (e.g., the i-th association character word) of the drug (e.g., dj) with reference to the large amount of literature information.

Here, D_k indicates the k-th literature information DB.

In FIG. 1, for convenience of description, one literature information DB 200 is illustrated, but there may be multiple literature information DBs 200.

The first matrix configuration unit 130 configures a drug-drug similarity matrix by calculating a cosine similarity between association word vectors of respective drugs on the basis of the configured association word vector of each drug.

For example, the first matrix configuration unit 130 may calculate a cosine similarity between the association word vector (T_dx) of an x-th drug and the association word vector (T_dy) of a y-th drug on the basis of information collected from the k-th literature information DB according to Equation 2 below, and then may configure a drug-drug similarity matrix indicating drug-drug similarity ranking on the basis of the calculation.

The drug-drug similarity ranking is generated for each k-th literature information DB 200, and the final drug-drug similarity matrix may be configured by using a value obtained by calculating an arithmetic mean of drug-drug similarity rankings generated for each k-th literature information DB 200.

$${D}_{k} ( cos ( {Td}_{x} , {Td}_{y} ) ) = \frac{{\sum }_{i} {t}_{ix} {t}_{iy}}{\sqrt{{\sum }_{i} {t}_{ix}^{2}} \sqrt{{\sum }_{i} {t}_{iy}^{2}}} \qquad [ Equation 2 ]$$

The first matrix configuration unit 130 configures an association word vector which indicates an appearance frequency of an association character word as an information value for each disease on the basis of the character information of the disease, the information being extracted from the literature information.

In addition, the first matrix configuration unit 130 may configure a disease-disease similarity matrix by calculating a cosine similarity between association word vectors of respective diseases on the basis of the association word vector of each disease.

Specifically, the first matrix configuration unit 130 may configure an association word vector for each disease on the basis of the character information of the disease, the information being extracted from the literature information, and, for example, the association word vector (T_dj) of the j-th disease (dj) may be represented as below.

T_dj={t_1j, t_2j, . . . , t_ij, . . . , t_nj}

Here, a value of t_ij indicates an information value in the association word vector of the disease (dj), and is defined to indicate an appearance frequency of the i-th association character word with respect to the disease (dj).

In this case, the information value (t_ij) in the association word vector is defined as a value obtained by normalizing a frequency count (T_ij) of the use (or appearances) of the i-th association character word of the disease (dj) in one piece of literature to a frequency count (n_i) of the use (or appearances) of the i-th association character word in all of the literature information, and may be represented according to Equation 1 above.

The information value (e.g., t_ij) in the association word vector of each disease may be defined as an appearance frequency (value) obtained by normalizing the appearance frequency of the association character word (e.g., the i-th association character word) of the disease (e.g., dj) with reference to the large amount of literature information.

Here, D_k indicates the k-th literature information DB.

The first matrix configuration unit 130 configures a disease-disease similarity matrix by calculating a cosine similarity between association word vectors of respective diseases on the basis of the configured association word vector of each disease.

For example, the first matrix configuration unit 130 may calculate a cosine similarity between the association word vector (T_dx) of the x-th disease and the association word vector (T_dy) of the y-th disease on the basis of information collected from the k-th literature information DB according to Equation 2 above, and then may configure a disease-disease similarity matrix indicating disease-disease similarity ranking on the basis of the calculation.

The disease-disease similarity ranking is generated for each k-th literature information DB 200, and the final drug-drug similarity matrix may be configured by using a value obtained by calculating an arithmetic mean of disease-disease similarity rankings generated for each k-th literature information DB 200.

As described above, the first matrix configuration unit 130 may configure a drug-drug or a disease-disease similarity matrix.

The second matrix configuration unit 140 performs a function of configuring a drug-drug or a disease-disease similarity matrix on the basis of information extracted from genomic signatures.

The second matrix configuration unit 140 configures a drug-drug or a disease-disease similarity matrix on the basis of the genetic association information of the drug and the disease, the information being extracted from the genomic signatures by the extraction unit 120.

An algorithm for configuring a drug-drug or a disease-disease similarity matrix on the basis of the genetic association information of the drug and the disease by the second matrix configuration unit 140 may be selected from any algorithm developed or used to infer a new drug repositioning candidate through mining of the existing drug-disease genetic association (response) data, and used.

In an embodiment for facilitating understanding of the present disclosure, each value in the drug-drug or the disease-disease similarity matrix configured by the second matrix configuration unit 140, that is, a semantic similarity score (similarity value) between drug- or disease-related genes, may be quantified according to the semantic similarity measure (Resnik et al., 1999), and accordingly, the similarity score (similarity value) may be modified in the range of [0, 1] by the rank normalization.

The calculation unit 150 may calculate a literature information-based drug-disease edge score (P_t) according to the similarity matrix configured by the first matrix configuration unit 130.

In addition, the calculation unit 150 may calculate a genomic signature-based drug-disease edge score (P_g) according to the similarity matrix configured by the second matrix configuration unit 140.

According to a specific process of calculating the drug-disease edge score (P_t), the calculation unit 150 may calculate a literature information-based drug-disease edge score (P_t) by using the similarity matrix configured by the first matrix configuration unit 130 and the drug-disease bipartite network configured by the network configuration unit 110.

Specifically, according to an embodiment, with respect to a pair of a particular drug (s_i, the i-th drug) and a particular disease (t_j, the j-th disease), the calculation unit 150 may calculate a drug-disease edge score (P_t) by using a similarity value between the particular drug (s_i) identified from the drug-drug similarity matrix configured by the first matrix configuration unit 130 and a reference drug (s_p) selected for calculation, a similarity value between the particular disease (t_j) identified from the disease-disease similarity matrix configured by the first matrix configuration unit 130 and a reference disease (t_q) selected for calculation, an edge between the reference drug (s_p) and the reference disease (t_q), and a degree value of the reference drug (s_p) identified from the drug-disease bipartite network.

Here, the pair of the particular drug (s_i, the i-th drug) and the particular disease (t_j, the j-th disease) corresponds to a query pair (a drug-disease pair, the edge score of which is to be identified), and may be a drug-disease pair specified (e.g., input as information) in order to identify recommendability.

Alternatively, the pair of the particular drug (s_i, the i-th drug) and the particular disease (t_j, the j-th disease) may be each of all drug-disease pairs obtained by automatically combining and matching the known drugs and the known diseases, respectively, in order to identify recommendability with respect to all the known drugs.

In other words, with respect to the pair of the particular drug (s_i) and the particular disease (t_j), the calculation unit 150 may calculate the drug-disease edge score (P_t) according to Equation 3 below.

Pₜ={square root over ((Sim LAB_{S}(sᵢ,sₚ)·Sim LAB_{T}(tⱼ,t_{q})))}·L(e_{pq})·w(sₚ)w(sₚ)=1−e^{-log10(D(s}^{ₚ}⁾⁾ [Equation 3]

Here, the particular drug (s_i) should belong to a set of all the known drugs (N_s) (si∈Ns), the particular disease (t_j) should belong to a set of all the known diseases (N_t) (tj∈Nt), and, similarly, the reference drug (s_p) and the reference disease (t_q) should belong to N_s and N_t, respectively (sp∈Ns, tq∈Nt).

SimLAB_s(s_i, s_p) is a similarity value (similarity ranking) between a particular drug (s_i) node identified from the drug-drug similarity matrix configured by the first matrix configuration unit 130 and a reference drug (s_p) node, and SimLAB_t(t_i, t_q) is a similarity value (similarity ranking) between a particular disease (t_j) node identified from the disease-disease similarity matrix configured by the first matrix configuration unit 130 and a reference disease (t_q) node.

L(e_pq) indicates the property (value) of an edge for connecting the reference drug (s_p) and the reference disease (t_j), and may be obtained by using a database representing the already known/verified drug-disease association relationships.

w(s_p) indicates a degree value of the reference drug (s_p) identified from the drug-disease bipartite network configured by the network configuration unit 110.

As noted from Equation 3, the value of the degree w(s_p) of the drug (s_p) node is determined by the number of first neighbor nodes of diseases (D(s_p)) connected by the edge in the drug (s_p) node in the drug-disease bipartite network.

Here, the reference drug (s_p) used to calculate the drug-disease edge score (P_t) for the particular drug (s_i) may be selected with reference to the pre-verified similarity to the particular drug (s_i) (e.g., the top rank of the similarity ranking), and the reference disease (t_q) having a true value of an edge label with the above-selected reference drug (s_p) from pre-verified drug-disease association relationships may be selected to be used to calculate the drug-disease edge score (P_t) for the particular drug (s_i).

Alternatively, the reference disease (t_q) used to calculate the drug-disease edge score (P_t) for the particular drug (s_i) may be selected with reference to the pre-verified similarity to the particular disease (t_j) (e.g., the top rank of the similarity ranking) that is paired with the particular drug (s_i) as a query pair, and the reference drug (s_p) having a true value of an edge label with the above-selected reference disease (t_q) from pre-verified drug-disease association relationships may be selected to be used to calculate the drug-disease edge score (P_t) for the particular drug (s_i).

Next, according to a specific process of calculating the drug-disease edge score (P_g), the calculation unit 150 may calculate a genomic signature-based drug-disease edge score (P_g) by using the similarity matrix configured by the second matrix configuration unit 140 and the drug-disease bipartite network configured by the network configuration unit 110.

Specifically, according to an embodiment, with respect to a pair of a particular drug (s_i) and a particular disease (t_j), the calculation unit 150 may calculate a drug-disease edge score (P_g) by using a similarity value between the particular drug (s_i) identified from the drug-drug similarity matrix configured by the second matrix configuration unit 140 and a reference drug (s_p) selected for calculation, a similarity value between the particular disease (t_j) identified from the disease-disease similarity matrix configured by the second matrix configuration unit 140 and a reference disease (t_q) selected for calculation, an edge between the reference drug (s_p) and the reference disease (t_q), and a degree value of the reference drug (s_p) identified from the drug-disease bipartite network.

Here, the pair of the particular drug (s_i) and the particular disease (t_j) is identical to the target query pair used to calculate the literature information-based drug-disease edge score (P_t).

Accordingly, with respect to the pair of the particular drug (s_i) and the particular disease (t_j), the calculation unit 150 may calculate the drug-disease edge score (P_g) according to Equation 4 below.

P_{g}={square root over ((Sim LAB_{S}(sᵢ,sₚ)·Sim LAB_{T}(tⱼ,t_{q})))}·L(e_{pq})·w(sₚ)w(sₚ)=1−e^{-log10(D(s}^{ₚ}⁾⁾ [Equation 4]

Here, the particular drug (s_i) should belong to a set of all the known drugs (N_s) (si∈Ns), the particular disease (t_j) should belong to a set of all the known diseases (N_t) (tj∈Nt), and, similarly, the reference drug (s_p) and the reference disease (t_q) should belong to N_s and N_t, respectively (sp∈Ns, tq∈Nt).

SimLAB_s(s_i, s_p) is a similarity value (similarity ranking) between a particular drug (s_i) node identified from the drug-drug similarity matrix configured by the second matrix configuration unit 140 and a reference drug (s_p) node, and SimLAB_t(t_i, t_q) is a similarity value (similarity ranking) between a particular disease (t_j) node identified from the disease-disease similarity matrix configured by the second matrix configuration unit 140 and a reference disease (t_q) node.

L(e_pq) indicates the property (value) of an edge for connecting the reference drug (s_p) and the reference disease (t_j), and may be obtained by using a database representing the already known/verified drug-disease association relationships.

w(s_p) indicates the degree value of the reference drug (s_p) identified from the drug-disease bipartite network configured by the network configuration unit 110.

As noted from Equation 4, the value of the degree w(s_p) of the drug (s_p) node is determined by the number of first neighbor nodes of diseases (D(s_p)) connected by the edge in the drug (s_p) node in the drug-disease bipartite network.

Here, the pair of the reference drug (s_p) and the reference disease (t_q) used to calculate the drug-disease edge score (P_g) for the particular drug (s_i) is identical to the drug-disease pair selected/used to calculate the literature information-based drug-disease edge score (P_t).

The final calculation unit 160 may calculate a final prediction score f(e_ij) of the drug-disease edge for the pair of the particular drug (s_i) and the particular disease (t_j), i.e., the current query pair, by using the score (P_t) and the score (P_g) calculated by the calculation unit 150.

Specifically, the final calculation unit 160 may identify heritability (H\hat{ }2 or h\hat{ }2) for the pair of the particular drug (s_i) and the particular disease (t_j), i.e., the current query pair, used to calculate the score (P_t) and the score (P_g) by the calculation unit 150.

When calculating the final prediction score f(e_ij) of the drug-disease edge, the score being calculated using the score (P_t) and the score (P_g) calculated by the calculation unit 150 for the current query pair (the pair of the drug (s_i) and the drug (t_j)), the final calculation unit 160 may calculate the final prediction score f(e_ij) by using different schemes according to the identified heritability.

According to an embodiment, when the identified heritability has a value equal to or larger than a predefined reference value (e.g., k heritability), the final calculation unit 160 may calculate the final prediction score f(e_ij) of the drug-disease edge by giving a larger weight to the genomic signature-based drug-disease edge score (P_g) than to the literature information-based drug-disease edge score (P_t).

For example, when the heritability has a value equal to or larger than a reference value of k, the final calculation unit 160 may calculate the final prediction score f(e_ij) of the drug-disease edge for the current query pair (the pair of the drug (s_i) and the disease (t_j)) according to Equation 5 below.

f(eᵢⱼ)=P_{g}/cos(P_{g}(eᵢⱼ)−Pₜ(eᵢⱼ)) [Equation 5]

On the other hand, when the identified heritability has a value smaller than the reference value (e.g., k heritability), the final calculation unit 160 may calculate the final prediction score f(e_ij) of the drug-disease edge by giving a larger weight to the literature information-based drug-disease edge score (P_t) than to the genomic signature-based drug-disease edge score (P_g).

For example, when the heritability has a value smaller than a reference value of k, the final calculation unit 160 may calculate the final prediction score f(e_ij) of the drug-disease edge for the current query pair (the pair of the drug (s_i) and the disease (t_j)) according to Equation 6 below.

f(eᵢⱼ)=Pₜ/cos(Pₜ(eᵢⱼ)−P_{g}(eᵢⱼ)) [Equation 6]

The recommendation unit 170 may recommend a drug repositioning candidate according to a value determined with reference to the final prediction score f(e_ij) calculated by the final calculation unit 160.

Specifically, the recommendation unit 170 may determine that the value is true (true=1) when the final prediction score f(e_ij) calculated by the final calculation unit 160 for the current query pair (the pair of the drug (s_i) and the disease (t_j)) is larger than a predefined threshold (θ), and may determine that the value is false (false=0) when the final prediction score f(e_ij) is not larger than the predefined threshold (θ).

The recommendation unit 170 may recommend the drug (s_i) of the current query pair as a drug repositioning candidate for the disease (t_j) when the value (true or false) determined with reference to the final prediction score f(e_ij) calculated by the final calculation unit 160 and the threshold (θ) is true (true=1).

As described above, according to the drug repositioning candidate recommendation system of the present disclosure, a new type of drug repositioning candidate recommendation technique (technology) for: representing a drug-indication relationship as a graph network model; quantifying/configuring a drug-drug and a disease-disease similarity matrix on the basis of literature information and genomic signatures, wherein the literature information and the genomic signatures are a large amount of big data; predicting a new indication of the drug on the basis of the quantified and configured matrices; and recommending a drug repositioning candidate according to the result of the new indication prediction of the drug, can be implemented.

According to the present disclosure, predicting a new indication of a drug, the safety of which has been verified, and recommending the drug, are possible through various drug- and disease-associated academic articles/literature information and genomic signatures that have been accumulated to date, without utilizing data which is inevitably restricted due to the lack of human resources and the characteristics of physiological information collected from human-derived materials of actual patients or symptom information or personal medical information protected by laws relating to personal information, whereby a significant reduction in drug development duration and cost can be expected.

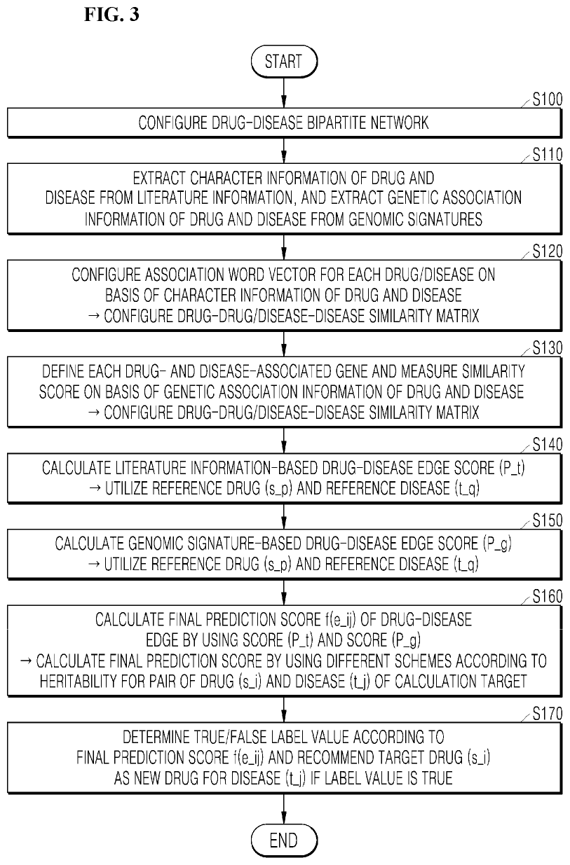

Hereinafter, referring to FIG. 3, a drug repositioning candidate recommendation technique (technology) according to an embodiment of the present disclosure is described.

The drug repositioning candidate recommendation technique (technology) of the present disclosure is implemented by a computer program according to an embodiment of the present disclosure, the computer program being stored in a medium so as to execute the operations described below.

For convenience of description, the drug repositioning candidate recommendation system 100 is described as an entity performing the operations.

According to the drug repositioning candidate recommendation technique of the present disclosure, the drug repositioning candidate recommendation system 100 configures a drug-disease bipartite network on the basis of drug indication information (operation S100).

Specifically, the drug repositioning candidate recommendation system 100 may configure a drug-disease bipartite network defined by set E={e_11, . . . , e_ij, . . . , e_mn} of N_s, N_t, and e_ij on the basis of the already known/verified drug indication information, i.e., by modeling drug-disease association relationships as a bipartite network.

In addition, according to the drug repositioning candidate recommendation technique (technology) of the present disclosure, the drug repositioning candidate recommendation system 100 may extract character information of a drug and a disease on the basis of open literature information, and extract genetic association information of a drug and a disease on the basis of genomic signatures (operation S110).

Specifically, the drug repositioning candidate recommendation system 100 may extract character information of a drug and a disease from literature information, which is a large amount of big data, on the basis of association with a literature information DB 200.

Here, the literature information may include at least one of: academic articles and medical or pharmaceutical books including description of symptoms of a disease, drug administration information, and description of a drug responsive character, a drug indication, or an adverse drug effect; an open database in which computational technology-based drug and disease association character information is collected; or disease and drug association description information.

The drug repositioning candidate recommendation system 100 may extract character information (an indication, an adverse effect, or a clinical phenotype) of a drug and a disease from a large amount of literature and bibliographic data such as academic articles, medical or pharmaceutical books, and disease- and drug-associated descriptive information.

In addition, the drug repositioning candidate recommendation system 100 may extract genetic association information of a drug and a disease from genomic signatures, which are a large amount of big data, on the basis of association with a genomic signature DB 300.

The drug repositioning candidate recommendation system 100 may collect and extract genetic association information (omics genomic information) from various large-scale genomic signatures (e.g., DrugBank, STITCH, OMIM, etc.) related to the drug and the disease.

According to the drug repositioning candidate recommendation technique of the present disclosure, the drug repositioning candidate recommendation system 100 configures a drug-drug or a disease-disease similarity matrix on the basis of the character information of the drug and the disease, the information being extracted from the literature information (operation S120).

Specifically, the drug repositioning candidate recommendation system 100 may configure an association word vector (T_dj) for each drug on the basis of the character information of the drug, the information being extracted from the literature information.

The drug repositioning candidate recommendation system 100 may configure a drug-drug similarity matrix by calculating association word vectors of respective drugs on the basis of the configured association word vector (T_dj) of each drug.

For example, the drug repositioning candidate recommendation system 100 may calculate a cosine similarity between the association word vector (T_dx) of an x-th drug and the association word vector (T_dy) of a y-th drug on the basis of information collected from the k-th literature information DB according to Equation 2 above, and then may configure a drug-drug similarity matrix indicating drug-drug similarity ranking on the basis of the calculation.

The drug-drug similarity ranking is generated for each k-th literature information DB 200, and the final drug-drug similarity matrix may be configured by using a value obtained by calculating an arithmetic mean of drug-drug similarity rankings generated for each k-th literature information DB 200.

The drug repositioning candidate recommendation system 100 configures an association word vector (T_dj) which indicates an appearance frequency of an association character word as an information value for each disease on the basis of the character information of the disease, the information being extracted from the literature information.

The drug repositioning candidate recommendation system 100 may configure a disease-disease similarity matrix by calculating a cosine similarity between association word vectors of respective diseases on the basis of the configured association word vector of each disease.

For example, the drug repositioning candidate recommendation system 100 may calculate a cosine similarity between the association word vector (T_dx) of the x-th disease and the association word vector (T_dy) of the y-th disease on the basis of information collected from the k-th literature information DB according to Equation 2 above, and then may configure a disease-disease similarity matrix indicating disease-disease similarity ranking on the basis of the calculation.

The disease-disease similarity ranking is generated for each k-th literature information DB 200, and the final disease-disease similarity matrix may be configured by using a value obtained by calculating an arithmetic mean of disease-disease similarity rankings generated for each k-th literature information DB 200.

Alternatively, according to the drug repositioning candidate recommendation technique of the present disclosure, the drug repositioning candidate recommendation system 100 configures a drug-drug or a disease-disease similarity matrix on the basis of the character information of the drug and the disease, the information being extracted from the genomic signatures (operation S130).

An algorithm for configuring a drug-drug or a disease-disease similarity matrix on the basis of the genetic association information of the drug and the disease in operation S130 may be selected from any algorithm developed or used to infer a new drug repositioning candidate through mining of the existing drug-disease genetic association (response) data, and used.

In an embodiment for facilitating understanding of the present disclosure, each value in the drug-drug or the disease-disease similarity matrix configured according to operation S130, that is, a semantic similarity score (similarity value) between drug- or disease-related genes, may be quantified according to the semantic similarity measure (Resnik et al., 1999), and accordingly, the similarity score (similarity value) may be modified in the range of [0, 1] by the rank normalization.

According to the drug repositioning candidate recommendation technique of the present disclosure, the drug repositioning candidate recommendation system 100 may calculate, in operation S140, a literature information-based drug-disease edge score (P_t) according to the similarity matrix configured in operation S120.

Specifically, the drug repositioning candidate recommendation system 100 may calculate a literature information-based drug-disease edge score (P_t) by using the similarity matrix configured in operation S120 and the drug-disease bipartite network configured in operation S100.

For example, with respect to a pair of a particular drug (s_i) and a particular disease (t_j), the drug repositioning candidate recommendation system 100 may calculate a drug-disease edge score (P_t) according to Equation 3 above by using a similarity value between the particular drug (s_i) identified from the drug-drug similarity matrix configured in operation S120 and a reference drug (s_p) selected for calculation, a similarity value between the particular disease (t_j) identified from the disease-disease similarity matrix configured in operation S120 and a reference disease (t_q) selected for calculation, an edge between the reference drug (s_p) and the reference disease (t_q), and a degree value of the reference drug (s_p) identified from the drug-disease bipartite network.

Here, the pair of the particular drug (s_i, the i-th drug) and the particular disease (t_j, the j-th disease) corresponds to a query pair (a drug-disease pair, the edge score of which is to be identified), and may be a drug-disease pair specified (e.g., input as information) to identify recommendability.

Alternatively, the pair of the particular drug (s_i, the i-th drug) and the particular disease (t_j, the j-th disease) may be each of all drug-disease pairs obtained by automatically combining and matching the known drugs and the known diseases, respectively, in order to identify recommendability with respect to all the known drugs.

Here, the reference drug (s_p) used to calculate the drug-disease edge score (P_t) for the particular drug (s_i) may be selected with reference to the pre-verified similarity to the particular drug (s_i) (e.g., the top rank of the similarity ranking), and the reference disease (t_q) having a true value of an edge label with the above-selected reference drug (s_p) from pre-verified drug-disease association relationships may be selected to be used to calculate the drug-disease edge score (P_t) for the particular drug (s_i).

Alternatively, the reference disease (t_q) used to calculate the drug-disease edge score (P_t) for the particular drug (s_i) may be selected with reference to the pre-verified similarity to the particular disease (t_j) (e.g., the top rank of the similarity ranking) that is paired with the particular drug (s_i) as a query pair, and the reference drug (s_p) having a true value of an edge label with the above-selected reference disease (t_q) from pre-verified drug-disease association relationships may be selected to be used to calculate the drug-disease edge score (P_t) for the particular drug (s_i).

In addition, according to the drug repositioning candidate recommendation technique of the present disclosure, the drug repositioning candidate recommendation system 100 may calculate, in operation S150, a genomic signature-based drug-disease edge score (P_g) according to the similarity matrix configured in operation S130.

Specifically, the drug repositioning candidate recommendation system 100 may calculate a genomic signature-based drug-disease edge score (P_g) by using the similarity matrix configured in operation S130 and the drug-disease bipartite network configured in operation S100.

For example, with respect to a pair of a particular drug (s_i) and a particular disease (t_j), the drug repositioning candidate recommendation system 100 may calculate a drug-disease edge score (P_g) according to Equation 4 above by using a similarity value between the particular drug (s_i) identified from the drug-drug similarity matrix configured in operation S130 and a reference drug (s_p) selected for calculation, a similarity value between the particular disease (t_j) identified from the disease-disease similarity matrix configured in operation S130 and a reference disease (t_q) selected for calculation, an edge between the reference drug (s_p) and the reference disease (t_q), and a degree value of the reference drug (s_p) identified from the drug-disease bipartite network.

Here, the pair of the particular drug (s_i) and the particular disease (t_j) is identical to the target query pair used to calculate the literature information-based drug-disease edge score (P_t).

The pair of the reference drug (s_p) and the reference disease (t_q) used to calculate the drug-disease edge score (P_g) for the particular drug (s_i) is identical to the drug-disease pair selected/used to calculate the literature information-based drug-disease edge score (P_t).

In addition, according to the drug repositioning candidate recommendation technique of the present disclosure, the drug repositioning candidate recommendation system 100 may calculate, in operation S160, a final prediction score f(e_ij) of the drug-disease edge for the pair of the particular drug (s_i) and the particular disease (t_j), i.e., the current query pair, by using the score (P_t) and the score (P_g) calculated in operations S140 and S150.

Specifically, the drug repositioning candidate recommendation system 100 may identify heritability (H\hat{ }2 or h\hat{ }2) for the pair of the particular drug (s_i) and the particular disease (t_j), i.e., the current query pair, used to calculate the score (P_t) and the score (P_g).

When calculating the final prediction score f(e_ij) of the drug-disease edge, the score being calculated using the score (P_t) and the score (P_g) calculated for the current query pair (the pair of the drug (s_i) and the drug (t_j)), the drug repositioning candidate recommendation system 100 may calculate the final prediction score f(e_ij) by using different schemes according to the identified heritability (operation S160).

According to an embodiment, when the identified heritability has a value equal to or larger than a predefined reference value (e.g., k heritability), the drug repositioning candidate recommendation system 100 may calculate the final prediction score f(e_ij) of the drug-disease edge by giving a larger weight to the genomic signature-based drug-disease edge score (P_g) than to the literature information-based drug-disease edge score (P_t).

For example, when the heritability has a value equal to or larger than a reference value of k, the drug repositioning candidate recommendation system 100 may calculate the final prediction score f(e_ij) of the drug-disease edge for the current query pair (the pair of the drug (s_i) and the disease (t_j)) according to Equation 5 above (operation S160).

On the other hand, when the identified heritability has a value smaller than the reference value (e.g., k heritability), the drug repositioning candidate recommendation system 100 may calculate the final prediction score f(e_ij) of the drug-disease edge by giving a larger weight to the literature information-based drug-disease edge score (P_t) than to the genomic signature-based drug-disease edge score (P_g).

For example, when the heritability has a value smaller than a reference value of k, the drug repositioning candidate recommendation system 100 may calculate the final prediction score f(e_ij) of the drug-disease edge for the current query pair (the pair of the drug (s_i) and the disease (t_j)) according to Equation 6 above (operation S160).

According to the drug repositioning candidate recommendation technique of the present disclosure, the drug repositioning candidate recommendation system 100 may recommend, in operation S170, a drug repositioning candidate according to a value determined with reference to the final prediction score f(e_ij) calculated in operation S160.

Specifically, the drug repositioning candidate recommendation system 100 may determine that the value is true (true=1) when the final prediction score f(e_ij) calculated for the current query pair (the pair of the drug (s_i) and the disease (t_j)) is larger than a predefined threshold (θ), and may determine that the value is false (false=0) when the final prediction score f(e_ij) is not larger than the predefined threshold (θ).

The drug repositioning candidate recommendation system 100 may recommend the drug (s_i) of the current query pair as a drug repositioning candidate for the disease (t_j) when the value (true or false) determined with reference to the calculated final prediction score f(e_ij) and the threshold (θ) is true (true=1).

As described above, according to the drug repositioning candidate recommendation technique (technology) of the present disclosure, a new type of drug repositioning candidate recommendation technique (technology) for: representing a drug-indication relationship as a graph network model; quantifying/configuring a drug-drug and a disease-disease similarity matrix on the basis of literature information and genomic signatures, respectively, wherein the literature information and the genomic signatures are a large amount of big data; predicting a new indication of the drug on the basis of the quantified and configured matrix; and recommending a drug repositioning candidate according to the result of the new indication prediction of the drug, can be implemented.

According to the present disclosure, predicting a new indication of a drug, the safety of which has been verified, and recommending the drug, are possible through various drug- and disease-associated academic articles/literature information and genomic signatures that have been accumulated to date, without utilizing data which is inevitably restricted due to the lack of human resources and the characteristics of physiological information collected from human-derived materials of actual patients or symptom information or personal medical information protected by laws relating to personal information, whereby a significant reduction in drug development duration and cost can be expected.

The drug repositioning candidate recommendation technique (technology) according to embodiments of the present disclosure may be implemented as a program command that can be executed by various computer means and may be recorded on a computer-readable medium. The computer-readable storage medium may include a program command, a data file, and a data structure, solely or in combination. The program command recorded on the medium may have been specially designed and configured for the present disclosure, or may be known to and available to those skilled in the field of computer software. Examples of the computer-readable storage medium include hardware devices specially configured to store and execute a program command, including magnetic media such as a hard disk, a floppy disk, and magnetic tape, optical media such as compact disk (CD)-read only memory (ROM) and a digital versatile disk (DVD), magneto-optical media such as a floptical disk, ROM, random access memory (RAM), and flash memory. Examples of the program command include not only a machine code such as a code generated by a compiler but also a high-level language code executable by a computer using an interpreter and the like. These hardware devices may be configured to operate as one or more software modules in order to perform the operation of the present disclosure, and the vice versa.

The present disclosure has been described in detail with reference to various embodiments, but is not limited to the embodiments, and those skilled in the art will appreciate that various changes or modifications without departing from the scope of the present disclosure as defined in the appended claims belong to the technical spirit of the present disclosure.

What is claimed is:

1. A drug repositioning candidate recommendation system, comprising:
an extraction unit configured to extract character information of a drug and a disease on the basis of open literature information, and extract genetic association information of a drug and a disease on the basis of genomic signatures;
a first matrix configuration unit configured to configure a drug-drug or a disease-disease similarity matrix on the basis of the information extracted from the literature information;
a second matrix configuration unit configured to configure a drug-drug or a disease-disease similarity matrix on the basis of the information extracted from the genomic signatures;
a calculation unit configured to calculate a literature information-based drug-disease edge score (P_t) according to the similarity matrix configured by the first matrix configuration unit, and calculate a genomic signature-based drug-disease edge score (P_g) according to the similarity matrix configured by the second matrix configuration unit; and
a recommendation unit configured to recommend a drug repositioning candidate according to a value determined by using at least one of the calculated score (P_t) and the calculated score (P_g).

2. A computer program stored in a medium so as to, in combination with hardware, execute:
an information extraction operation of extracting character information of a drug and a disease on the basis of open literature information, and extracting genetic association information of a drug and a disease on the basis of genomic signatures;
a first matrix configuration operation of configuring a drug-drug or a disease-disease similarity matrix on the basis of the information extracted from the literature information;
a second matrix configuration operation of configuring a drug-drug or a disease-disease similarity matrix on the basis of the information extracted from the genomic signatures;
a calculation operation of calculating a literature information-based drug-disease edge score (P_t) according to the similarity matrix configured in the first matrix configuration operation, and calculating a genomic signature-based drug-disease edge score (P_g) according to the similarity matrix configured in the second matrix configuration operation; and
a recommendation operation of recommending a drug repositioning candidate according to a value determined by using at least one of the calculated score (P_t) and the calculated score (P_g).

3. The computer program of claim 2, wherein the recommendation operation comprises:
a final calculation operation of calculating a final prediction score f(e_ij) of a drug-disease edge by using the calculated score (P_t) and the calculated score (P_g); and
a recommendation operation of recommending a drug repositioning candidate according to a value determined with reference to the final prediction score f(e_ij).

4. The computer program of claim 2, wherein the literature information comprises at least one of: academic articles and medical or pharmaceutical books comprising description of symptoms of a disease, drug administration information, and description of a drug responsive character, a drug indication, or an adverse drug effect; an open database in which character information associated with drug and disease is collected based on computational technology; and description information associated with drug and disease.

5. The computer program of claim 2, wherein the first matrix configuration operation comprises:
configuring an association word vector which indicates an appearance frequency of an association character word as an information value for each drug on the basis of the character information of a drug extracted from the literature information; and
configuring a drug-drug similarity matrix by calculating a cosine similarity between association word vectors of respective drugs on the basis of the association word vector of each drug.

6. The computer program of claim 2, wherein the first matrix configuration operation comprises:
configuring an association word vector which indicates an appearance frequency of an association character word as an information value for each disease on the basis of the character information of a disease extracted from the literature information; and
configuring a disease-disease similarity matrix by calculating a cosine similarity between association word vectors of respective diseases on the basis of the association word vector of each disease.

7. The computer program of claim 5, wherein an information value in the association word vector of the drug or an information value in the association word vector of the disease is defined as t_ij indicating an appearance frequency of an i-th association character word of a j-th drug or a j-th disease, and
the information value (t_ij) is a value obtained by normalizing a frequency count (T_ij) of appearances of the i-th association character word in one piece of literature for a frequency count (n_i) of appearances of the i-th association character word in all of the literature information.

8. The computer program of claim 6, wherein an information value in the association word vector of the drug or an information value in the association word vector of the disease is defined as t_ij indicating an appearance frequency of an i-th association character word of a j-th drug or a j-th disease, and
the information value (t_ij) is a value obtained by normalizing a frequency count (T_ij) of appearances of the i-th association character word in one piece of literature for a frequency count (n_i) of appearances of the i-th association character word in all of the literature information.

9. The computer program of claim 2, wherein the computer program is configured to further execute a network configuration operation of configuring a drug-disease bipartite network on the basis of drug indication information, and
wherein the calculation operation comprises:
calculating a literature information-based drug-disease edge score (P_t) by using the similarity matrix configured in the first matrix configuration operation and the configured drug-disease bipartite network; and
calculating, with respect to a pair of a particular drug (s_i, an i-th drug) and a particular disease (t_j, a j-th disease), a drug-disease edge score (P_t) by using a similarity value between the particular drug (s_i) identified from the drug-drug similarity matrix configured in the first matrix configuration operation and a reference drug (s_p) selected for calculation, a similarity value between the particular disease (t_j) identified from the disease-disease similarity matrix configured in the first matrix configuration operation and a reference disease (t_q) selected for calculation, an edge between the reference drug (s_p) and the reference disease (t_q), and a degree value of the reference drug (s_p) identified from the drug-disease bipartite network.

10. The computer program of claim 9, wherein the reference drug (s_p) is selected with reference to a pre-verified similarity to the particular drug (s_i), and the reference disease (t_q) having a true value of an edge label with the reference drug (s_p) from pre-verified drug-disease association relationships is selected, or
the reference disease (t_q) is selected with reference to a pre-verified similarity to the particular disease (t_j), and the reference drug (s_p) having a true value of an edge label with the reference disease (t_q) from pre-verified drug-disease association relationships is selected.

11. The computer program of claim 3, wherein the final calculation operation comprises:
identifying heritability with respect to a pair of a particular drug (s_i) and a particular disease (t_j) used to calculate the score (P_t) and the score (P_g); and
calculating the final prediction score f(e_ij) of the drug-disease edge differently depending on the heritability.

12. The computer program of claim 11, wherein the final calculation operation comprises:
calculating, when the heritability has a value equal to or larger than a predefined reference value, the final prediction score f(e_ij) of the drug-disease edge by giving a larger weight to the genomic signature-based drug-disease edge score (P_g) than to the score (P_t); and
calculating, when the heritability has a value smaller than the reference value, the final prediction score f(e_ij) of the drug-disease edge by giving a larger weight to the literature information-based drug-disease edge score (P_t) than to the score (P_g).

13. The computer program of claim 3, wherein the recommendation operation comprises:
determining a true or false value according to a cut-off; and
identifying, when the value is true, a pair of a particular drug (s_i) and a particular disease (t_j) used to calculate the final prediction score f(e_ij) so as to recommend the particular drug (s_i) as a new drug for the particular disease (t_j).
